(19) United States
(12) Patent Application Publication
(10) Pub. No.: US 2008/0233063 A1
(43) Pub. Date: Sep. 25, 2008

(54) LUSTROUS AND SCRATCH-RESISTANT NAIL VARNISH THROUGH ADDITION OF SOL-GEL SYSTEMS

(76) Inventors: Heike Bergandt, Marl (DE); Frank Weinelt, Billerbeck (DE)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/037,431

(22) Filed: Feb. 26, 2008

(30) Foreign Application Priority Data
Feb. 26, 2007 (DE) 102007009590.4

(51) Int. Cl. A61K 8/30; A61Q 3/02

(52) U.S. Cl. 424/61

(57) ABSTRACT

The present invention relates to, for example, a nail varnish additive, a nail varnish additive composition for improving at least one property of nail varnishes, the nail varnish additive composition being, in one embodiment, in the form of a sol-gel system; to a process for forming a nail varnish additive composition; to a process for forming a nail varnish comprising a nail varnish additive composition; to a nail varnish comprising a nail varnish additive composition; to a process for forming a laminate comprising a nail and the nail varnish additive composition; and to a laminate comprising a nail and the nail varnish additive.

### CROSS REFERENCE TO RELATED APPLICATION

This application claims priority to German Patent Application Number DE 102007009590.4, filed on Feb. 26, 2007. German Patent Application Number DE 102007009590.4 is incorporated, by reference, in its entirety.

### BACKGROUND OF THE INVENTION

1. Field of the Invention

The present invention relates to, for example, a nail varnish additive, a nail varnish additive composition for improving at least one property of nail varnishes, the nail varnish additive composition being, in one embodiment, in the form of a sol-gel system; to a process for forming a nail varnish additive composition; to a process for forming a nail varnish comprising a nail varnish additive composition; to a nail varnish comprising a nail varnish additive composition; to a process for forming a laminate comprising a nail and the nail varnish additive composition; and to a laminate comprising a nail and the nail varnish additive.

2. Discussion of the Background

A modern nail varnish serves to maintain and convey a pleasing shape and colouration of fingernails and toenails. Additionally, the nail varnish protects the nail against environmental influences and the nail gains high hardness from the varnish. Special efforts have been undertaken to make applied nail varnishes long-lasting, scratch-resistant, splinterproof, attractive in colour and pleasing in lustre.

Current nail varnishes comprise a large number of ingredients. The most important representatives of these include the film formers, adhesion promoters, plasticizers, solvents and pigments. Fumed silicas are used for modifying the rheology and thixotropy of nail varnishes. Even with these ingredient options, however, the shelf-life, hardness and scratch resistance of current nail varnishes are still unsatisfactory.

The primary film former used in current nail varnishes is nitrocellulose. Nitrocellulose ensures some degree of good hardness and toughness of the varnish surface. However, as is disclosed in DE 32 43 291 A1, further resins generally have to be added in order to reduce fracture and chipping.

U.S. Pat. No. 4,873,077, GB 1177420 and DE 69111621 disclose various additives, in the form of synthetic, natural, and mineral fibres, for improving wear resistance, combating nail splitting, and avoiding nail cracking, chipping and/or fracture.

DE 433-4938 A1 describes that alkyl-branched and polyglycol-ether-containing fatty acid esters are recommended for rapid drying with hard and more scratch-resistant films.

For improving the external appearance, the application DE 198 22 722 A1 discloses inorganic-organic hybrid polymers which can be added to a nail varnish formulation. These are organofunctional silanes which carry three condensable and hydrolysable functions on the silicon atom, and also have a crosslinkable organic radical in the molecule. Drying temperatures of 40 to 50° C. are specified as particularly preferred, and the drying time should be 2 to 20 min.

Copolymers of acrylates and methacrylates, disclosed in FR 7614430, and copolymers with polar functions, disclosed in DE 31 12 888 C2, are said to reduce brittleness of the dried nail varnish and improve its adhesion to the nail.

It is common to all these above listed documents that, to improve the properties of nail varnishes, a large number of substances are added and then admixed to the finished cosmetic product in an expensive process.

In view of the sensitivity of consumers toward side-effects arising from nail varnish use, said sensitivity having been on the increase for a long time, it must also be observed that a consumer will refrain completely from the use of nail varnishes if the consumer has to reckon with allergy-triggering effects due to additives such as, for example, admixed acrylate monomers.

It is therefore one object of the present invention to provide, for example, a nail varnish additive composition which can be admixed easily into a nail varnish and which overcomes the above described disadvantages.

### DETAILED DESCRIPTION OF THE PREFERRED EMBODIMENTS

Surprisingly, it has been found that nail varnish is improved through the simple addition of a nail varnish additive which has

a) at least one reaction product arising out of a composition comprising
a1) at least of one silane of the general formula

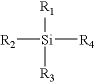

where the radicals R₁, R₂, R₃, R₄ are identical or different having 1 to 40 carbon atoms and are non-crosslinking, and
a2) a silane of the general formula

Si(OR′)₄,
where the radical R′ has 1 to 40 carbon atoms and is a non-crosslinking alkoxy group, and

b) at least one solvent, and

c) water, and

d) a catalyst.

The present invention likewise provides a process for producing a composition by combining the following components:

a1) in an amount from 0 to 75% by weight, and

a2) in an amount from 0 to 75% by weight, and

b) in an amount from 10 to 50% by weight, and

c) in an amount from 0 to 15% by weight, and

d) in an amount from 0 to 5% by weight,

the quantitative data being based in each case on the composition, and with the proviso that the sum of the parts by weight is 100%, or comprises these components.

The present invention also provides for a composition comprising

a1) in an amount from 0 to 75% by weight, and

a2) in an amount from 0 to 75% by weight, and

b) in an amount from 10 to 50% by weight, and

c) in an amount from 0 to 15% by weight, and

d) in an amount from 0 to 5% by weight,

the quantitative data being based in each case on the composition, and with the proviso that the sum of the parts by weight is 100%, or comprises these components.

The present invention also provides a process for producing a nail varnish additive composition wherein a composition is produced which contains component

a1) in an amount from 0 to 75% by weight, and

a2) in an amount from 0 to 75% by weight, and

b) in an amount from 10 to 50% by weight, and

c) in an amount from 0 to 15% by weight, and

d) in an amount from 0 to 5% by weight,

by combining these components to produce the composition,

wherein, upon combining, in addition to components a1)-d) being present in the composition, at least one reaction product arising from the combination of a1)-d) is also present in the composition, the quantitative data being based in each case on the composition, and with the proviso that the sum of the parts by weight is 100%, or comprises these components.

The present invention also provides for a nail varnish additive composition comprising

a1) in an amount from 0 to 75% by weight, and

a2) in an amount from 0 to 75% by weight, and

b) in an amount from 10 to 50% by weight, and

c) in an amount from 0 to 15% by weight, and

d) in an amount from 0 to 5% by weight, and

e) at least one reaction product arising from the combination of components a1)-d)

The present invention likewise provides a nail varnish additive and/or nail varnish additive composition which is obtained by the process according to the invention.

The present invention also provides a nail varnish containing a nail varnish additive and/or nail varnish additive composition according to the invention, and a process for making a nail varnish containing a nail varnish additive and/or nail varnish additive composition according to the invention comprising blending the nail varnish additive and/or the nail varnish additive composition, with a nail varnish to form the nail varnish containing the nail varnish additive and/or nail varnish additive composition.

The nail varnish and nail varnish additive composition according to the invention may be in liquid form, for example, as found in a container or when initially applied to a nail, and can also be, for example, in sol-gel form.

The nail varnish, nail varnish additive, and/or nail varnish additive composition according to the invention may be also be in dried form, for example, after being applied to a nail and allowed to dry/cure.

The present invention has the advantage that the nail varnish admixed with the nail varnish additive and/or nail varnish additive composition according to the invention dries to give a highly lustrous and brilliant coating on the nail.

The drying time of the nail varnish admixed with the nail varnish additive and/or nail varnish additive composition according to the invention is comparable with the drying time of conventional nail varnishes.

The nail varnish additive according to the invention has the advantage that, following application to the nail surface, during hardening, the silanes in the nail varnish additive and/or nail varnish additive composition react with the moisture available in the air via hydrolysis and condensation.

For the user there is the further advantage that the nail varnish with the nail varnish additive and/or nail varnish additive composition according to the invention can be applied to the nails without its user being required to readapt, for example in the sense of longer waiting times or special application techniques out of the ordinary.

Besides rapid drying, adhesion to the nail and the ability to be removed from the nail surface is a critical product property. Comparative tests with conventional nail varnishes not of the invention have surprisingly shown that an inventive varnish provided with the inventive nail varnish additive and/or nail varnish additive composition according to the invention adheres just as well, or better, to the nail surface when compared to the conventional nail varnishes not of the invention, but nevertheless can be removed using conventional nail varnish remover just as easily and rapidly as dried conventional nail varnish not of the invention.

A particular advantage of the nail varnish additive and/or nail varnish additive composition according to the invention is that the nail varnishes admixed therewith have higher hardness and better scratch resistance than conventional nail varnishes not of the invention that do not contain the inventive nail varnish additive and/or nail varnish additive composition.

Furthermore, besides the high hardness, a certain flexibility of the varnished nail surface is also important in order to allow movements of the nail with it and thereby to have the lowest possible tendency for chipping and fracture. Nail varnish admixed with nail varnish additive and/or nail varnish additive composition according to the invention has the advantage that the dried nail varnish, compared with a conventional nail varnish not of the invention, chips or fractures less, if at all.

The present invention therefore also provides a nail varnish containing the nail varnish additive and/or nail varnish additive composition according to the invention; a method of forming a nail varnish containing the inventive nail varnish additive and/or nail varnish additive composition comprising combining the nail varnish with the inventive nail varnish additive and/or nail varnish additive composition to form the nail varnish containing the inventive nail varnish additive and/or inventive nail varnish additive composition; and a method of forming a laminate comprising a nail and a nail varnish comprising an inventive nail varnish additive and/or nail varnish additive composition comprising contacting/applying an inventive nail varnish to the nail to form the laminate.

Furthermore, an advantage of the present invention is that the proportion of film-forming components such as nitrocellulose and/or polyacrylates, which can have an allergic sensitizing effect, in a nail varnish, can be reduced as a result of employing an inventive nail varnish additive and/or nail varnish additive composition (e.g., by adding an inventive nail varnish additive and/or nail varnish additive composition to a nail varnish, the amount of film forming components can be reduced or eliminated).

While the performance profile even of a conventional nail varnish can be significantly improved as a result of adding the nail varnish additive and/or nail varnish additive composition according to the invention, a further advantage of the present invention is that no undesired competing reactions of any kind with the components found in a conventional nail varnish arise.

It is a very particular advantage of the present invention that the proportion of these components with which hardness, lustre, flexibility and scratch resistance of the conventional nail varnish are usually achieved in conventional nail varnishes be reduced or eliminated. Hardness, lustre, brilliance, flexibility and scratch resistance are achieved merely as a result of adding the nail varnish additive and/or nail varnish additive composition according to the invention to a nail varnish, and even improved compared with the features of conventional nail varnishes.

The present invention therefore likewise provides a nail varnish which contains or is the nail varnish additive and/or nail varnish additive composition according to the invention which hardens following application to the nail as a result of reaction with atmospheric moisture. The present invention therefore also further provides the use of the nail varnish according to the invention for cosmetics and/or nail care (e.g., applying a nail varnish according to the invention to, for example, a nail).

The nail varnish according to the invention has the advantage of better protecting the nail against impacts and environmental influences as result of the simultaneously improved flexibility and hardness. Fracture and chipping of the nails are likewise advantageously reduced.

The present invention is described below by reference to an example without any intention to restrict the invention, the protective scope of which arises from the claims and the description, thereto. The claims themselves also belong to the disclosure content of the present invention. Where the text below states ranges or preferred ranges, then all theoretically possible part ranges within these ranges should also belong to the disclosure content of the present invention without these having been explicitly specified for reasons of better clarity.

The present invention provides a nail varnish additive, wherein the nail varnish additive has

a) at least one reaction product arising out of a composition comprising
a1) at least of one silane of the general formula

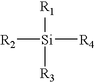

where the radicals R₁, R₂, R₃, R₄ are identical or different having 1 to 40 carbon atoms and are non-crosslinking, and
a2) a silane of the general formula

Si(OR′)₄,
where the radical R′ has 1 to 40 carbon atoms and is a non-crosslinking alkoxy group, and

b) at least one solvent, and

c) water, and

d) a catalyst.

The present invention provides a nail varnish additive composition, wherein the nail varnish additive composition comprises
a1) of at least of one silane of the general formula

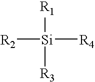

where the radicals R₁, R₂, R₃, R₄ are identical or different having 1 to 40 carbon atoms and are non-crosslinking, and
a2) a silane of the general formula

Si(OR′)₄,
where the radical R′ has 1 to 40 carbon atoms and is a non-crosslinking alkoxy group, and

b) at least one solvent, and

c) water, and

d) a catalyst, and

e) at least one reaction product arising from the combination of ingredients a1)-d)

It may be advantageous if the radicals R₁, R₂, R₃, R₄ of component a1) of, for example, the nail varnish additive and/or nail varnish additive composition according to the invention are selected from straight-chain, branched or alicyclic alkyl, fluoroalkyl, aryl, alkoxy groups, or a combination of these groups. Particular preference is given to selecting alkoxy groups. Preferably, the alkoxy groups of the nail varnish additive according to the invention can be selected from methoxy, ethoxy, propoxy, butoxy, particularly preferably selected from ethoxy, methoxy, propoxy, very particularly preferably ethoxy, methoxy, or a combination of these groups.

Furthermore, it may be advantageous if in component a2) the radical R′ of, for example, the nail varnish additive and/or nail varnish additive composition according to the invention is selected from methoxy, ethoxy, propoxy, or butoxy, particularly preferably selected from ethoxy, methoxy, propoxy, very particularly preferably ethoxy, methoxy, or a combination of these groups.

Preferably, component b) of, for example, the nail varnish additive and/or nail varnish additive composition according to the invention can be selected from the series of alcohols of the general formula CₙH₂ₙ₊₁OH, where n=1 to 4, particularly preferably n=2 or 3, from the series of polyhydric alcohols, from ketones, acetates, glycol ethers, toluene, xylene, or from a mixture of these solvents. This component can particularly preferably be selected from glycols, acetone, methyl ethyl ketone, ethyl acetate, butyl acetate, ethylene glycol monomethyl ether, or a mixture of these solvents.

Preferably, component d) of, for example, the nail varnish additive and/or nail varnish additive composition according to the invention can be selected from hydrochloric acid, nitric acid, phosphoric acid, sulphuric acid, formic acid, propionic acid, acetic acid, citric acid, or a mixture of these acids. This component can particularly preferably be selected from hydrochloric acid, nitric acid, or phosphoric acid, very particularly preferably from phosphoric acid, nitric acid, or a mixture of these acids.

It may be advantageous if the nail varnish additive and/or nail varnish additive composition according to the invention, for example, has at least one reaction product a3) at least of a further silane of the general formula

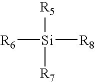

where the radicals R₅, R₆, R₇, R₈ are identical or different having 1 to 40 carbon atoms, straight-chain, branched or alicyclic fluoroalkyl, alkylurethanofluoroalkyl groups, and are noncrosslinking. Preferably, the radicals R₅, R₆, R₇, R₈ of component a3) can be selected from a combination of these groups. Component a3) can particularly preferably be selected from Dynasilan F8261, available from Degussa GmbH, Paul-Baumann-Strasse 1, 45764 Marl.

The present invention likewise provides a process for producing a nail varnish additive composition, characterized in that a composition is produced which contains component

a1) in an amount from 0.1 to 75% by weight, and
a2) in an amount from 0.1 to 75% by weight, and
b) in an amount from 10 to 50% by weight, and
c) in an amount from 0.1 to 15% by weight, and
d) in an amount from 0.1 to 5% by weight, and
e) a reaction product arising from the combination of ingredients a1)-d),
the quantitative data being based in each case on the composition, and with the proviso that the sum of the parts by weight is 100%, or comprises these components.

Preferably, in the process according to the invention, component a1) can be used in an amount from 0.15 to 74.9% by weight, 0.5 to 74.9% by weight, 0.75 to 74.9% by weight, 1.0% by weigh to 74.9% by weight, 5% by weight to 74.9% by weight, 10% by weight to 74.9% by weight, 15% by weight to 74.9% by weight, 20% by weight to 74.9% by weight, 25% by weight to 74.9% by weight, 30% by weight to 74.9% by weight, 35% by weight to 74.9% by weight, 40% by weight to 74.9% by weight, 45% by weight to 74.9% by weight, 50% by weight to 74.9% by weight, 55% by weight to 74.9% by weight, preferably from 0.5 to 60% by weight, more preferably from 1 to 50% by weight, further preferably from 5 to 50% by weight, particularly preferably from 10 to 40% by weight, very particularly preferably from 20 to 30% by weight, and a2) can preferably be used in an amount from 0.15 to 74.9% by weight, 0.5 to 74.9% by weight, 0.75 to 74.9% by weight, 1.0% by weigh to 74.9% by weight, 5% by weight to 74.9% by weight, 10% by weight to 74.9% by weight, 15% by weight to 74.9% by weight, 20% by weight to 74.9% by weight, 25% by weight to 74.9% by weight, 30% by weight to 74.9% by weight, 35% by weight to 74.9% by weight, 40% by weight to 74.9% by weight, 45% by weight to 74.9% by weight, 50% by weight to 74.9% by weight, 55% by weight to 74.9% by weight, preferably from 0.5 to 60% by weight, more preferably from 1 to 50% by weight, further preferably from 5 to 50% by weight, particularly preferably from 10 to 40% by weight, very particularly preferably from 20 to 30% by weight, and b) can preferably be used in an amount from 12 to 48% by weight, 15% by weight to 48% by weight, 20% by weight to 48% by weight, 25% by weight to 48% by weight, 30% by weight to 48% by weight, 35% by weight to 48% by weight, 40% by weight to 48% by weight, 45% by weight to 48% by weight, particularly preferably from 15 to 40% by weight, very particularly preferably from 18 to 35% by weight, and c) can preferably be used in an amount from 0.15 to 11.9% by weight, 0.2% by weight to 11.9% by weight, 0.3% by weight to 11.9% by weight, 0.4% by weight to 11.9% by weight, 0.5% by weight to 11.9% by weight, 0.6% by weight to 11.9% by weight, 0.7% by weight to 11.9% by weight, 0.8% by weight to 11.9% by weight, 0.9% by weight to 11.9% by weight, 1% by weight to 11.9% by weight, 2% by weight to 11.9% by weight, 3% by weight to 11.9% by weight, 4% by weight to 11.9% by weight, 5% by weight to 11.9% by weight, 6% by weight to 11.9% by weight, 7% by weight to 11.9% by weight, 8% by weight to 11.9% by weight, 9% by weight to 11.9% by weight, 10% by weight to 11.9% by weight, preferably from 0.2 to 12% by weight, more preferably from 0.5 to 10% by weight, particularly preferably from 1 to 8% by weight, very particularly preferably from 2 to 5% by weight, and d) can preferably be used in an amount from 0.15 to 4.9% by weight, preferably from 0.2 to 4.5% by weight, more preferably from 0.5 to 4% by weight, particularly preferably from 1 to 3% by weight, very particularly preferably from 1.5 to 2% by weight, the quantitative data being based in each case on the composition, and with the proviso that the sum of the weight fractions is 100%.

In the process according to the invention, a dilute or concentrated acid, preferably from 0.5 to 10% strength, 0.6% strength, 0.7% strength, 0.8% strength, 0.9% strength, 1.0% strength, 2.0% strength, 3.0% strength, 4.0% strength, 5.0% strength, 6.0% strength, 7.0% strength, 8.0% strength, 9.0% strength, particularly preferably from 1 to 7% strength, very particularly preferably from 1.5% to 5% strength acid can preferably be used as component d).

It may be advantageous if, in the process according to the invention, components a1), a2), b), c) and d) are mixed by stirring. Preferably, in the process according to the invention, a silane, particularly preferably component a1), can be initially introduced, and then components b), c), d), finally a2), further preferably firstly component a2), then b), c), d) and finally a1), are added. Preferably, each component can be initially introduced and the other components be added in any desired order, very particularly preferably b) is initially introduced, then c), a1), a2) and d) are added.

In the process according to the invention, it may furthermore be advantageous if a composition is produced which contains the further component a3) in an amount from 0 to 2% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight, 1.6% by weight, 1.7% by weight, 1.8% by weight, preferably in an amount from 0.1 to 1.9% by weight, particularly preferably in an amount from 0.2 to 1.5% by weight, very particularly preferably in an amount from 0.5 to 1.5% by weight, the quantitative data being based in each case on the composition, and with the proviso that the sum of the weight fractions of components a1), a2), a3), b), c), and d) is 100%.

In the process according to the invention, the components can be particularly preferably mixed together by stirring in a propeller stirrer, oblique-blade stirrer, disc stirrer, impeller stirrer, cross-blade stirrer, anchor stirrer, blade stirrer, gate stirrer, helical stirrer, toothed-disc stirrer, turbine stirrer, half-moon stirrer, or fan stirrer. It may be advantageous if, in the process according to the invention, stirring techniques are used which introduce little or no ambient gas into the composition and/or in which little heat energy is introduced into the composition. In the process according to the invention, propeller stirrers, oblique-blade stirrers, disc stirrers, impeller stirrers, cross-blade stirrers, anchor stirrers, blade stirrers, gate stirrers, helical stirrers, or toothed-disc stirrers can be used very particularly preferably, furthermore propeller stirrers, disc stirrers or impeller stirrers can be used very particularly preferably.

Preferably, in the process according to the invention, components a1), b), c), d) and a2), particularly preferably components a1), a2), b), c), d) and a3) with low shear rates can be mixed together over a period of from 1 to 120 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, preferably from 15 to 100 min, particularly preferably from 30 to 90 min, very particularly preferably from 45 to 75 min, extraordinarily particularly preferably from 55 to 65 min. It may be advantageous if, in the process according to the invention, the components of the composition are mixed together for a sufficiently long period for the amount of component c) to drop to a fraction from 0.01 to 10% by weight, 0.02% by weight, 0.03% by weight, 0.04% by weight, 0.05% by weight, 0.06% by weight, 0.07% by weight, 0.08% by weight, 0.09% by weight, 0.1% by weight, 0.2% by weight, 0.3% by weight, 0.4% by weight, 0.5% by weight, 0.6% by weight, 0.7% by weight, 0.8% by weight, 0.9% by weight, 1% by weight, 2% by weight, 3% by weight, 4% by weight, 5% by weight, 6% by weight, 7% by weight, 8% by weight, 9% by weight, 10% by weight, preferably to a fraction from 0.05 to 8% by weight, particularly preferably to a fraction from 0.1 to 5% by weight, further particularly preferably from 0.2 to 3% by weight, very particularly preferably from 0.3 to 2% by weight, extraordinarily particularly preferably from 0.5 to 1.9% by weight.

Preferably, in the process according to the invention, the components of the nail varnish additive according to the invention can be mixed at a temperature from 1 to 30° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C. 28° C., 29° C., particularly preferably from 5 to 28° C., further particularly preferably from 10 to 25° C., very particularly preferably from 18 to 21.5° C., extraordinarily preferably at room temperature by stirring.

The present invention likewise provides the use of the nail varnish additive and or nail varnish additive composition according to the invention in a nail varnish (e.g., a varnish containing the nail varnish additive and/or nail varnish additive composition according to the invention, and a method of making a varnish containing the nail varnish additive and/or nail varnish additive composition according to the invention, comprising combining the varnish with the nail varnish additive and/or nail varnish additive composition).

It may be advantageous if the nail varnish additive and/or nail varnish additive composition according to the invention is used in a concentration from 1 to 13% by weight, 2% by weight, 3% by weight, 4% by weight, 5% by weight, 6% by weight, 7% by weight, 8% by weight, 9% by weight, 10% by weight, 11% by weight, 12% by weight, preferably from 1.5 to 12% by weight, particularly preferably from 2 to 11% by weight, very particularly preferably from 2.5 to 10% by weight, in the nail varnish. For the purposes of the present invention, the weight data are based on the weight of the nail varnish ready for the user and prepared for application to the nails. In one advantageous embodiment of the use according to the invention, the nail varnish additive and/or nail varnish additive composition according to the invention can be incorporated into this nail varnish by stirring or shaking with the nail varnish, which may be a conventional nail varnish or a nail varnish without film-forming components. The use according to the invention has the advantage that the dried nail varnish applied to the nail simultaneously has lustre, brilliance, hardness, wear resistance and flexibility. If preferably as component a1) and/or a2) Dynasylan A, Dynasylan TEOS, Dynasilan MTES, trimethylethoxysilane, phenylethyltriethoxysilane, and/or for component a3) Dynasilan F8261, all of these silanes available from Degussa GmbH, Paul-Baumann-Strasse 1, 45764 Marl, or a mixture of these silanes is used, the use according to the invention furthermore has the advantage that the resulting nail varnish retains the cosmetically intended shade, compared with the shade of conventional nail varnish, meaning that reformulation with regard to the colour design is not necessary.

The present invention is described by way of example in the examples hereinafter. Obviously, numerous modifications and variations of the present invention are possible in light of the above teachings. It is therefore to be understood that, within the scope of the appended claims, the invention may be practiced otherwise than as specifically described herein.

### Example 1

### Preparation of a Nail Varnish Additive Composition

10% by weight of Dynasylan TEOS, 50% by weight of Dynasylan MTES, 12% by weight of nitric acid in a concentration of 1%, 12% by weight of 2-propanol and 16% by weight of butyl acetate were mixed together with stirring at room temperature with low shear rates over a period of 60 min. After this time, the water content was reduced to 1.95% by weight, and at least one reaction product was formed after the above ingredients were combined.

10% by weight of the nail varnish additive composition obtained in this way was then added to 90% by weight of standard commercial nail varnish. The nail varnish additive composition was mixed thoroughly with the nail varnish by stirring at room temperature until a homogeneous distribution was achieved.

Drying of the nail varnish was accompanied by crosslinking and condensation of the silanes following application of the nail varnish to the nail surface as a result of the reaction with atmospheric moisture.

### Example 2

As Example 1, but using 50% by weight of Dynasylan TEOS, 15% by weight of Dynasylan MTES, 1% by weight of Dynasylan F8261, 12% by weight of phosphoric acid in a concentration of 1%, and 22% by weight of 2-propanol as nail varnish additive composition, wherein least one reaction product was formed, and contained in the nail varnish additive composition, after the above ingredients were combined.

### Example 3

As Example 2, but mixing 5% by weight of nail varnish additive composition with 95% by weight of standard commercial nail varnish.

### Example 4

As Example 2, but mixing 2.5% by weight of nail varnish additive composition with 97.5% by weight of standard commercial nail varnish.

### Example 5

As Example 1, but mixing the nail varnish additive composition with a nonconventional nail varnish. The nonconventional nail varnish had the composition according to Table 1.

TABLE 1

| | |
|---|---|
| 16 g | Nitrocellulose (Wolff Cellulosics GmbH & Co. KG) |
| 10 g | Arylsulphonamide |
| 6 g | Acetyl tributylcitrate, camphor |
| 1.5 g | Stearalkonium bentonite |
| 1.5 g | Pigments |
| 65 g | Butyl acetate |

### Example 6

As Example 1, but mixing the nail varnish additive composition with a nonconventional nail varnish. The nonconventional nail varnish had the composition according to Table 2.

TABLE 2

| | |
|---|---|
| 16.5 g | Nitrocellulose (Wolff Cellulosics GmbH & Co. KG) |
| 10.3 g | Arylsulphonamide |
| 6.2 g | Acetyl tributylcitrate, camphor |
| 67 g | Butyl acetate |

### Example 7

As Example 1, but mixing the nail varnish additive composition with a nonconventional nail varnish. The nonconventional nail varnish had the composition according to Table 3.

TABLE 3

| | |
|---|---|
| 10 g | Nitrocellulose (Wolff Cellulosics GmbH & Co. KG) |
| 6 g | Styrene/acrylate copolymer |
| 10 g | Sol-gel mixture from Example 1 |
| 6 g | Acetyl tributylcitrate, camphor |
| 1.5 g | Stearalkonium bentonite |
| 1.5 g | Pigments |
| 40 g | Butyl acetate |
| 25 g | Ethyl acetate |

### Example 8

As Example 1, but mixing the nail varnish additive composition with a nonconventional nail varnish. The nonconventional nail varnish had the composition according to Table 4.

TABLE 4

| | |
|---|---|
| 15 g | Nitrocellulose (Wolff Cellulosics GmbH & Co. KG) |
| 10 g | Styrene/acrylate copolymer |
| 10.3 g | Sol-gel mixture from Example 2 |
| 6.2 g | Acetyl tributylcitrate, camphor |
| 27 g | Butyl acetate |
| 31.5 g | Ethyl acetate |

### Example 9

As Example 1, but mixing the nail varnish additive composition with a nonconventional nail varnish. The nonconventional nail varnish had the composition according to Table 5.

TABLE 5

| | |
|---|---|
| 16 g | Nitrocellulose (Wolff Cellulosics GmbH & Co. KG) |
| 3 g | Sol-gel mixture from Example 1 |
| 6 g | Acetyl tributylcitrate, camphor |
| 1.5 g | Stearalkonium bentonite |
| 1.5 g | Pigments |
| 40 g | Butyl acetate |
| 32 g | Ethyl acetate |

### Example 10

As Example 1, but mixing the nail varnish additive composition with a nonconventional nail varnish. The nonconventional nail varnish had the composition according to Table 6.

TABLE 6

| | |
|---|---|
| 16.5 g | Nitrocellulose (Wolff Cellulosics GmbH & Co. KG) |
| 3.0 g | Sol-gel mixture from Example 2 |
| 6.2 g | Acetyl tributylcitrate, camphor |
| 40 g | Butyl acetate |
| 34.3 g | Ethyl acetate |

The above written description of the invention provides a manner and process of making and using it such that any person skilled in this art is enabled to make and use the same, this enablement being provided in particular for the subject matter of the appended claims, which make up a part of the original description of the invention.

As used above, the phrases “selected from the group consisting of,” “chosen from,” and the like include mixtures of specified materials.

All references, patents, applications, tests, standards, documents, publications, brochures, texts, articles, etc. mentioned herein are incorporated herein by reference. Where a numerical range or limit is stated, the endpoints are included. Also, all values and subranges within a numerical limit or range are specifically included as if explicitly written out. Terms such as “contain(s)” and the like as used herein are open terms meaning ‘including at least’ unless otherwise specifically noted. The terms “a” and “an” mean one or more, unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A nail varnish additive composition comprising
a) a1) at least of one silane of the general formula 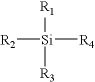
wherein the radicals R₁, R₂, R₃, R₄ are identical or different having 1 to 40 carbon atoms and are non-crosslinking, and
a2) a silane of the general formula
Si(OR′)₄,
wherein the radical R′ has 1 to 40 carbon atoms and is a non-crosslinking alkoxy group, and
b) at least one solvent, and
c) water, and
d) a catalyst, and
e) and at least one reaction product arising from the combination of elements a1)-d).

2. The nail varnish additive composition of claim 1, wherein the radicals R₁, R₂, R₃, R₄ are each independently selected from the group consisting of straight-chain alkyl group, a branched-chain alkyl group, an alicyclic alkyl group, a straight-chain fluoroalkyl group, a branched-chain fluoroalkyl group, an alicyclic fluoroalkyl group, an aryl group, an alkoxy group, a straight-chain alkylurethanofluoroalkyl group, a branched-chain alkylurethanofluoroalkyl group, and an alicyclic alkylurethanofluoroalkyl group.

3. The nail varnish additive composition of claim 1, wherein the radicals R₁, R₂, R₃, R₄ are identical or different and are selected from the group consisting of methoxy, ethoxy, propoxy, and butoxy.

4. The nail varnish additive composition of claim 1, wherein the radical R′ is selected from the group consisting of methoxy, ethoxy, propoxy, and butoxy.

5. The nail varnish additive composition of claim 1, wherein component b) is selected from the group consisting of an alcohol of the general formula CₙH₂ₙ₊₁OH, wherein n=1 to 4, a mixture of these alcohols, a polyhydric alcohol, a ketone, an acetate, a glycol ether, toluene, xylene, and a mixture of these solvents.

6. The nail varnish additive composition of claim 1, wherein component d) is selected from the group consisting of hydrochloric acid, nitric acid, phosphoric acid, sulphuric acid, formic acid, propionic acid, acetic acid, citric acid, and a mixture of these acids.

7. The nail varnish additive composition of claim 1,
which further comprises
a3) at least one further silane of the general formula 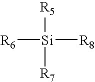
wherein the radicals R₅, R₆, R₇, R₈ are identical or different, have 1 to 40 carbon atoms, and are each selected from the group consisting of a straight-chain fluoroalkyl group, a straight-chain alkylurethanofluoroalkyl group, a branched-chain fluoroalkyl group, a branched-chain alkylurethanofluoroalkyl group, an alicyclic fluoroalkyl group, and an alicyclic alkylurethanofluoroalkyl group; wherein the radicals are non-crosslinking.

8. A process for producing the nail varnish additive composition of claim 1, comprising combining
a1) in an amount from 0.1 to 75% by weight, and
a2) in an amount from 0.1 to 75% by weight, and
b) in an amount from 10 to 50% by weight, and
c) in an amount from 0 to 15% by weight, and
d) in an amount from 0.1 to 5% by weight,
the amounts being based in each case on a total amount of the composition, with the proviso that the sum of the parts by weight of all components in the composition is 100%, thereby obtaining said nail varnish additive composition, wherein the composition further comprises at least one reaction product arising from the combination of ingredients a1)-d).

9. The process according to claim 8,
further comprising combining a component
a3) in an amount from 0 to 2% by weight,
the amounts being based in each case on the composition, and with the proviso that the sum of the weight fractions is 100%.

10. The process of claim 8, wherein the components are combined by stirring.

11. A nail varnish additive composition obtained by the process of claim 8.

12. A method of making a nail varnish comprising a nail varnish additive composition, the method comprising
combining a nail varnish with the nail varnish additive composition of claim 1 to form the nail varnish comprising the nail varnish additive composition.

13. A nail varnish which comprises the nail varnish additive composition of claim 1.

14. A method of caring for a nail, comprising applying the nail varnish additive composition of claim 1 to the nail.

15. The nail varnish additive composition of claim 2, wherein the radical R′ is selected from the group consisting of methoxy, ethoxy, propoxy, and butoxy.

16. The nail varnish additive composition of claim 2, wherein component b) is selected from the group consisting of an alcohol of the general formula CₙH₂ₙ₊₁OH, wherein n=1 to 4, a mixture of these alcohols, a polyhydric alcohol, a ketone, an acetate, a glycol ether, toluene, xylene, and a mixture of these solvents.

17. The nail varnish additive composition of claim 3, wherein component b) is selected from the group consisting of an alcohol of the general formula CₙH₂ₙ₊₁OH, wherein n=1 to 4, a mixture of these alcohols, a polyhydric alcohol, a ketone, an acetate, a glycol ether, toluene, xylene, and a mixture of these solvents.

18. The nail varnish additive composition of claim 4, wherein component b) is selected from the group consisting of an alcohol of the general formula CₙH₂ₙ₊₁OH, wherein n=1 to 4, a mixture of these alcohols, a polyhydric alcohol, a ketone, an acetate, a glycol ether, toluene, xylene, and a mixture of these solvents.

19. A process for producing the nail varnish additive composition of claim 1, comprising combining
a1) in an amount from 0.1 to 75% by weight, and
a2) in an amount from 0.1 to 75% by weight, and
b) in an amount from 10 to 50% by weight, and
c) in an amount from 0 to 15% by weight, and
d) in an amount from 0.1 to 5% by weight,
the amounts being based in each case on a total amount of the composition, thereby obtaining said nail varnish additive composition, wherein the composition further comprises at least one reaction product arising from the combination of ingredients a1)-d).

20. The process of claim 19, wherein the components are combined by stirring.
